(19) United States
(12) Patent Application Publication
(10) Pub. No.: US 2016/0128764 A1
(43) Pub. Date: May 12, 2016

(54) HIGH-FREQUENCY PROSTATE TREATMENT MACHINE

(71) Applicant: HUMANDREAM.CO.,LTD, Jeonju-si, Jeollabuk-do (KR)

(72) Inventors: Mokyung KIM, Jeonju-si, Jeollabuk-do (KR); Hyobum KIM, Jeonju-si, Jeollabuk-do (KR)

(21) Appl. No.: 14/897,091

(22) PCT Filed: Sep. 17, 2014
(86) PCT No.: PCT/KR2014/008634

§ 371 Date: Jan. 20, 2016

(30) Foreign Application Priority Data
Sep. 30, 2013 (KR) 10-2013-0115876

(51) Int. Cl. A61B 18/14

(52) U.S. Cl. CPC A61B 18/1485; A61B 18/1477; A61B 2018/00547

(57) ABSTRACT

The present invention relates to a high-frequency prostate treatment device, wherein an insertion unit, a high-frequency output unit, and an electrode unit, which are used for treating prostate, may be combined as one product, and the patient's prostate tissue may be treated more efficiently through a small number of components.

### TECHNICAL FIELD

The present invention relates to a high-frequency prostate treatment device, in which an insertion unit, a high-frequency output unit, and an electrode unit, which are used for treating prostate, are combined as one product and also the less number of components is used to more efficiently treat patient's prostate tissue.

### BACKGROUND ART

In general, the prostate is one of male sex-accessory organs that are capable of performing a reproductive function together with testicles.

The prostate produces about 35% of fluid components of semen, and the produced prostate fluid provides nutrition to the sperm and prevents the ejaculated semen from being hardened to increase the fluidity of the sperm, thereby increasing sperm fertility.

The prostate is a soft tissue disposed right below the bladder of men.

The prostate has a hole defined in a center thereof, and the urethra passes through the hole. Accordingly, the abnormal prostate causes problems in urination and also sexual dysfunction.

Representative examples of prostatic diseases may include prostatic hypertrophy, prostatitis, and prostate cancer.

The prostatic hypertrophy is a disease that occurs by fat compositions accumulated in the prostate tissue.

The prostatic hypertrophy causes difficulty in urination of men older than the middle age and also causes various urological problems. To treat the prostatic hypertrophy, various treatment methods have been developed and used. So far, there are three representative treatment methods such as thermotherapy, injection therapy, and physical therapy.

The prostate thermotherapy is a method in which heat having a predetermined range of temperature (about 40° C. to about 45° C.) is radiated into the prostate to release heat for treatment.

That is, although normal cells are dead at about 45° C., tumor cells are sensitive to heat, and thus dead at a lower temperature of about 42.5° C.

Thus, if the temperature is maintained between about 42.5° C. and 45° C., the normal cells are alive, whereas the tumor cells are dead.

Although the prostate thermotherapy is mainly used for treating abacterial prostatitis, the prostate thermotherapy may be combined for improving symptom in bacterial prostatitis in some cases.

In principle, although the prostate thermotherapy is used only for treating the abacterial prostatitis, the prostate thermotherapy may be used for improving symptom in bacterial prostatitis.

When the treatment at a temperature of about 43° C. for about 60 minutes is defined as one session,

it is sent that five to six session treatments improve symptom in about 66% of patients and

reduce the number of leukocyte in the prostate fluid in about 77% of the patients.

In recent years, a prostate treatment device that is capable of performing centesis of a treatment mechanism into a desired portion over an entire prostate area to treat the prostate disease through easy operation and also reducing the burden of the patient is disclosed in Japanese Patent Laid-Open No. 2000-271235.

However, in case of Japanese Patent Laid-Open No. 2000-271235 (prostate treatment device),

a large number of components having complex structures such as

a microwave radiation probe, an ultrasonic transreceiver, a balloon, an ultrasonic observer, and a microwave oscillator have to be used for the prostate treatment.

### DISCLOSURE OF THE INVENTION

### Technical Problem

To solve the above-described limitations,

the present invention is to provide a high-frequency prostate treatment device in which an insertion unit, a high-frequency output unit, and an electrode unit, which are used for treating prostate, are combined as one product, and

also the small number of components is used to more efficiently treating patient's prostate tissue.

### Technical Solution

Embodiments of the present invention provide high-frequency prostate treatment devices including: an insertion unit inserted into a prostate tissue through an anus; a high-frequency output unit inserted into the insertion unit to receive high-frequency current from a main body that generates the high-frequency current, thereby generating high-frequency deep heat; an electrode unit disposed on the insertion unit while being spaced a predetermined distance from the high-frequency output unit, the electrode unit having a potential opposite to that of the high-frequency output unit.

In some embodiments, the insertion unit may include: a coupling jig of which an upper portion is screw-coupled to the high-frequency output unit; a probe support in which an accommodation groove accommodating a lower portion of the coupling jig is defined in an upper portion thereof, and a through hole communicating with the accommodation groove vertically extends by a predetermined length so that the through hole is defined below the accommodation groove, the probe support being accommodated in the electrode unit having a tubular shape; and a fixing cap screw-coupled to a lower portion of the probe support.

In other embodiments, a temperature sensor may be disposed in the high-frequency output unit so that the temperature sensor is disposed above the coupling jig.

In still other embodiments, a through hole may be vertically and eccentrically defined in the coupling jig.

In even other embodiments, a groove having a predetermined depth may be defined

in a top surface of the coupling jig.

In yet other embodiments, an accommodation groove accommodating a lower portion of the probe support may be defined in an upper portion of the fixing cap, and

a through hole communicating with the accommodation groove may be defined in a lower portion of the fixing cap.

In further embodiments, the high-frequency prostate treatment device may further include: a temperature setting part setting a reference temperature value of the high-frequency output unit; and a timer setting a time range for generating high-frequency deep heat that is generated by the high-frequency output unit.

In still further embodiments, the high-frequency prostate treatment device may further include a cover surrounding the insertion unit, the high-frequency output unit, and the electrode unit.

### Advantageous Effects

According to the present invention, the high-frequency output unit and the electrode unit may be provided to the insertion unit to be inserted into the prostate tissue to allow the insertion unit, the high-frequency output unit, and the electrode unit to be combined as one product, and also, the patient's prostate tissue may be more efficiently treated by using the three components, i.e., the insertion unit, the high-frequency output unit, and the electrode unit.

### BRIEF DESCRIPTION OF THE DRAWINGS

FIG. 1 is a schematic plan view of a high-frequency prostate treatment device

according to an embodiment of the present invention.

FIG. 2 is a partially enlarged plan view of an insertion unit, a high-frequency output unit, and an electrode unit of FIG. 1.

FIG. 3 is an exploded cross-sectional view of FIG. 2.

FIG. 4 is a coupled cross-sectional view of FIG. 3.

FIG. 5 is a schematic plan view of a coupling jig in FIG. 3.

FIG. 6 is a schematic plan view of a cover in FIG. 6.

FIG. 7 is a cross-sectional view of a high-frequency treatment device, which has a structure different from that of the high-frequency treatment device of FIG. 3 according to another embodiment.

FIG. 8 is a coupled cross-sectional view of FIG. 7.

FIG. 9 is a view illustrating an example of a circuit generating high-frequency current.

FIG. 10 is a schematic flowchart illustrating a process for treating prostate of a patient.

### MODE FOR CARRYING OUT THE INVENTION

Hereinafter, the present invention will be described in detail with reference to the accompanying drawings. Moreover, detailed descriptions related to well-known functions or configurations will be ruled out in order not to unnecessarily obscure subject matters of the present invention.

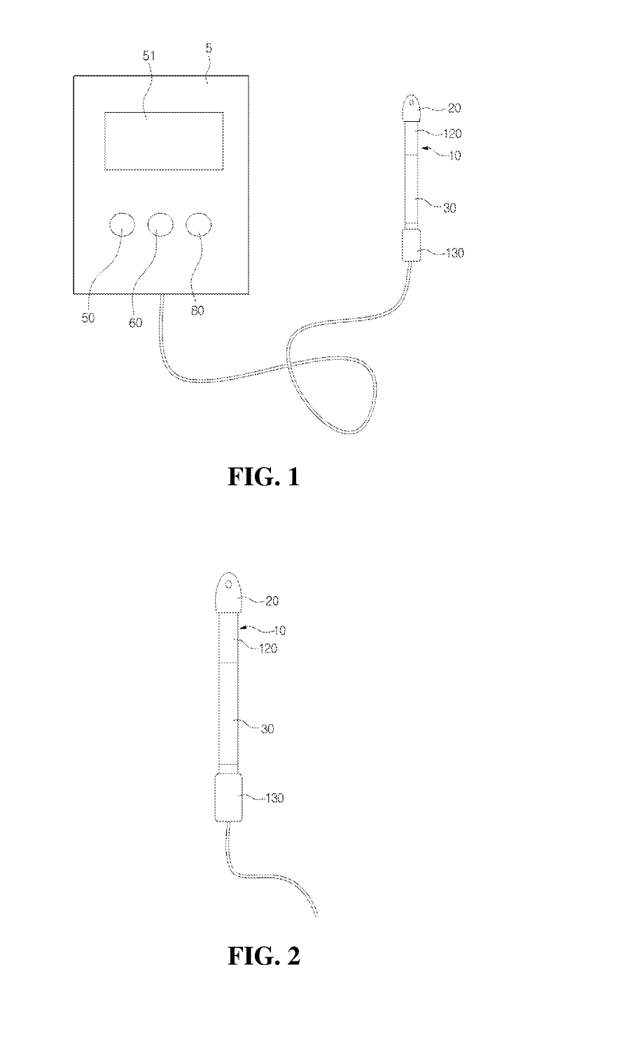

FIG. 1 is a schematic plan view of a high-frequency prostate treatment device according to an embodiment of the present invention, and

FIG. 2 is a partially enlarged plan view illustrating an insertion unit 10, a high-frequency output unit 20, and an electrode unit 30 in FIG. 1.

The high-frequency prostate treatment device according to an embodiment of the present invention includes

the insertion unit 10, the high-frequency output unit 20, and the electrode unit 30 as illustrated in FIGS. 1 and 2.

First, the insertion unit 10 may be inserted into a prostate tissue of a patient

through an anus of the patient by a professional therapist such as a doctor.

Then, the high-frequency output unit 20 may be provided to the insertion unit 10 and connected to a main body 5 generating high-frequency current through a connecting line such as an electric wire.

The main body 5 may generate the high-frequency current of about 0.1 MHz to about 10 MHz to more significantly increase efficiency in prostate disease treatment of the patient.

The high-frequency output unit 20 receives the high-frequency current from the main body 5

to generate high-frequency deep heat, thereby treating the prostate disease of the patient.

The high-frequency output unit 20 may be made of a conductive metallic material such as aluminum having an outer surface insulating-coated with ceramic to prevent an electric shock accident from occurring.

The technical idea of present invention may be applied to all of a capacity electric transfer (CET) method or/and a resistive electric transfer (RET) method.

Next, the electrode unit 30 is provided to the insertion unit 10

while being maintained at a predetermined distance from the high-frequency output unit 20.

The electrode unit 30 has a potential opposite to that of the high-frequency output unit 20.

The electrode unit 30 may be made of a conductive metallic material such as aluminum.

The high-frequency output unit 20 may be constituted by an electrode having a cap shape, and the electrode unit 30 may be formed by manufacturing an electrode plate in a tubular shape. The electrode constituting the high-frequency output unit 20 and the electrode plate constituting the electrode unit 30 and manufactured in the tubular shape are well known through Korea Registered Patent No. 10-1128607, and thus, their detailed descriptions will be omitted.

In the shape of the high-frequency output unit, if an initially inserted portion of the high-frequency treatment device is defined as an upper end, since the high-frequency output unit has a width that gradually decreases toward the upper end, the high-frequency output unit may be easily inserted, and also electric field intensity may be intensified to increase a high-frequency output. For example, the upper end of the high-frequency output unit has a width that is less than a transverse width of at least the electrode unit. As described above, the shape that is sharpened toward the upper end is based on the principle in which charges increases in density as an end of a conductor is sharpened. Thus, the frequency output from the high-frequency output unit may increase.

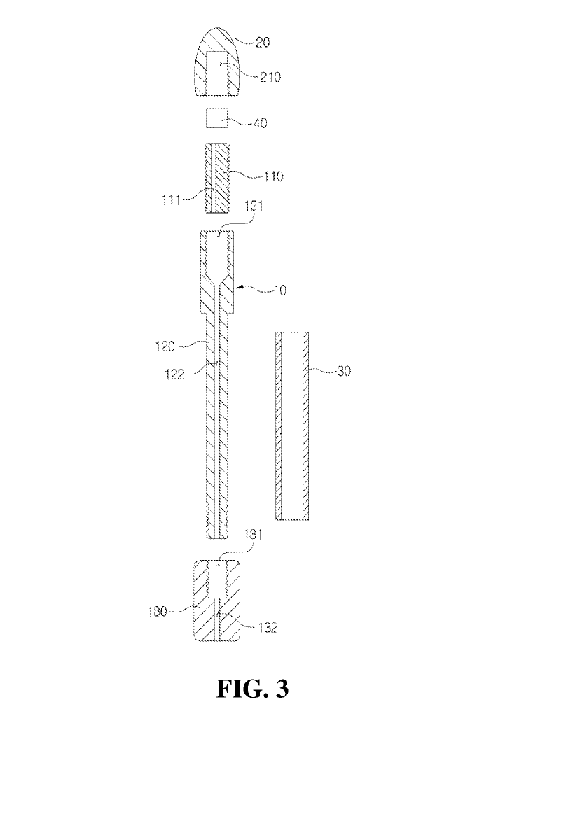

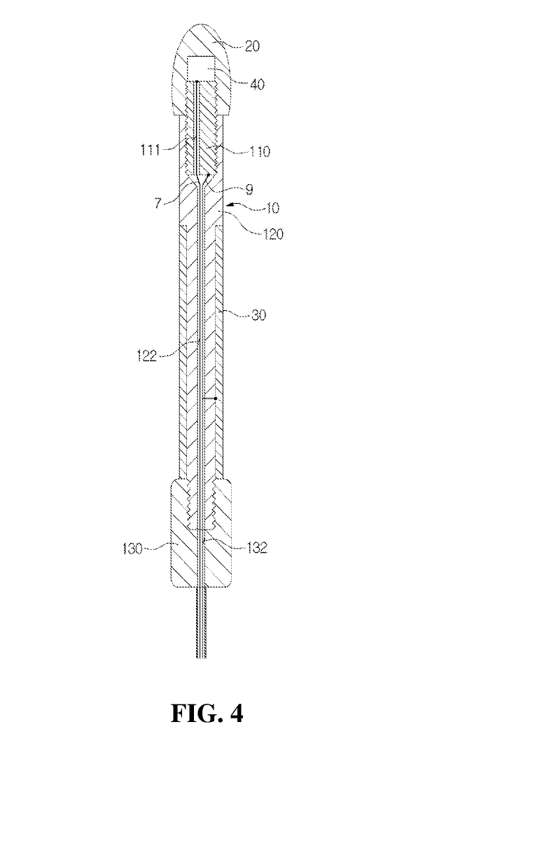

FIG. 3 is an exploded cross-sectional view of FIG. 2, and FIG. 4 is a coupled cross-sectional view of FIG. 3.

In more detail, the insertion unit 10 may include

a coupling jig 110, a probe support 120, and a fixing cap 130 as illustrated in FIGS. 3 and 4.

An accommodation groove 210 vertically extending from a lower portion of the high-frequency output unit 20 to an upper portion of the high-frequency output unit 20 and having an opened lower side may be defined in the high-frequency output unit 20.

An upper portion of the coupling jig 110 may be accommodated in a lower portion of an accommodation groove 210 of the high-frequency output unit 20

while being screw-coupled to an inner circumferential surface of the lower portion of the accommodation groove 210 of the high-frequency output unit 20.

The coupling jig 110 may be made of a conductive metallic material such as aluminum.

An accommodation groove 121 in which a lower portion of the coupling jig 110 is accommodated in a screw-coupled state may be defined

in an upper portion in the probe support 120.

A through hole 122 communicating with the accommodation groove 121 may be defined the probe support 120 to vertically extend by a predetermined length

from an upper portion of the probe support 120 to a lower portion of the probe support 120

so that the through hole 122 is defined below the accommodation groove 121 of the probe support 120.

The probe support 120 may be accommodated

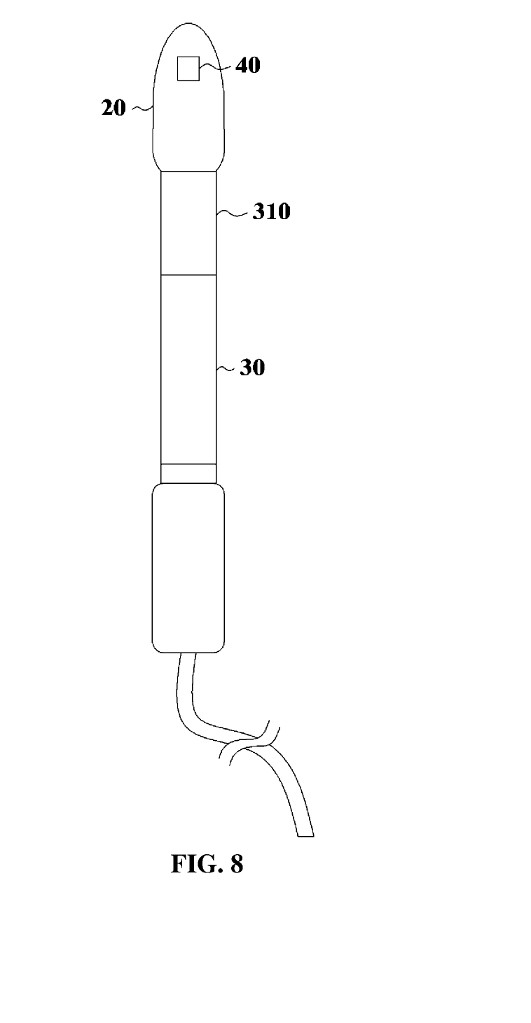

in the tubular-shaped electrode unit 30. Accordingly, even in a case in which only the electrode unit is mentioned according to the foregoing embodiment and another embodiment (see FIGS. 7 and 8) of the present invention, the present invention should be construed as including the probe support. The probe support may be included thereto. Especially, even the electrode unit of FIG. 7 may include the probe support described in FIG. 3.

The probe support 120 may be made of

an insulating material such as polyethylene (PE).

The fixing cap 130 may be screw-coupled to the lower portion of the probe support 120.

The fixing cap 130 may be made of

an insulating material such as rubber and polyethylene (PE).

An accommodation groove 131 in which the lower portion of the probe support 120 is accommodated in a screw-coupled state may be recessed by a predetermined depth from an upper portion of the fixing cap 130 to a lower portion of the fixing cap 130 in the upper portion of the fixing cap 130.

A through hole 132 communicating with the accommodation groove 131 may be defined in an upper portion of the fixing cap 130

to vertically extend by a predetermined length from the upper portion of the fixing cap 130 to the lower portion of the fixing cap 130

so that the through hole 132 is defined below the accommodation groove 131 of the fixing cap 130.

Next, as illustrated in FIGS. 3 and 4,

a temperature sensor 40 may be disposed in an upper portion of the accommodation groove 210 defined in the high-frequency output unit 20 so that the temperature sensor 40 is disposed above the coupling jig 110.

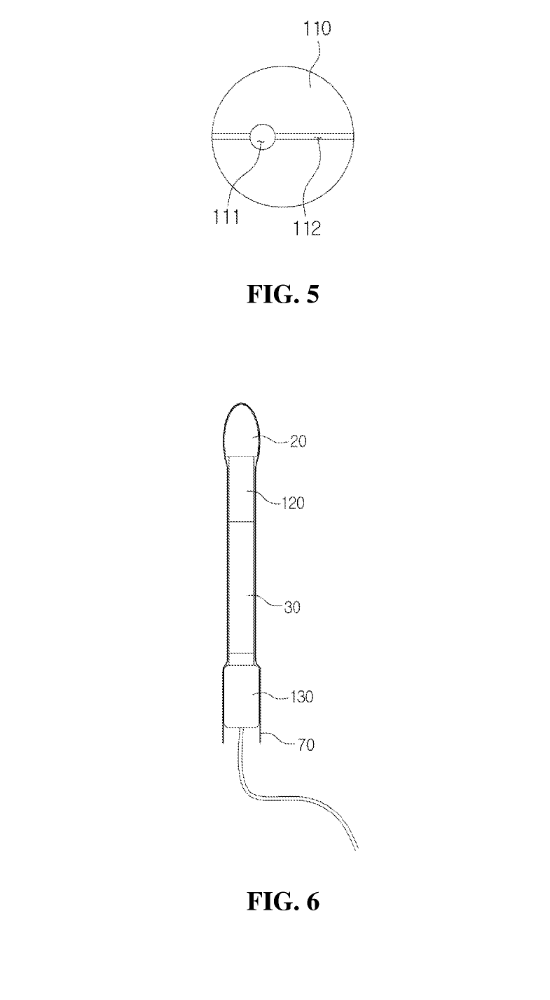

FIG. 5 is a schematic plan view of the coupling jig 110 of FIG. 3.

Next, as illustrated in FIGS. 3 to 5,

a through hole 110 vertically extending from an upper portion of the fixing jig 110 to a lower portion of the coupling jig 110 may be eccentrically defined in the coupling jig 110.

A through hole 111 of the coupling jig 110 may be, e.g., vertically and eccentrically defined

in one side of the coupling jig 110.

As illustrated in FIG. 4, a connecting line 7 such as an electric line, of which a lower portion is connected to the main body 5, may have an upper portion that sequentially passes through the through hole 132 of the fixing cap 130, the through hole 122 of the probe support 120, and

the through hole 111 of the coupling jig 110 and be connected to a lower portion of one side of the temperature sensor 40.

Also, another connecting line 9 such as an electric line, of which a lower portion is connected to the main body 5, may have

an upper portion that sequentially passes through the through hole 132 of the fixing cap 130, the through hole 122 of the probe support 120, and the through hole 111 of the coupling jig 110 and be connected to a lower portion of the other side of the coupling jig 110.

Here, since the through hole 111 of the coupling jig 110 is vertically and eccentrically defined in the one side of the coupling jig 110, the lower portion of the other side of the coupling jig 110 may have an area greater than that of the lower portion of the one side of the coupling jig 110, and thus an upper portion of the another connecting line 9 may be easily connected to the lower portion of the other side of the coupling jig 110. Additionally explaining, as illustrated in FIG. 4, since the through hole 111 is eccentric to a left side with respect to a center, a relatively large area (a right portion of the through hole and a right portion with respect to the center) may be used for other purposes.

The high-frequency current generated from the main body 5 may be sequentially applied to the coupling jig 110 and the high-frequency output unit 20 through the another connecting line 9

The another connecting line 9 may be branched to be connected to the tubular-shaped electrode unit 30

while horizontally passing through the probe support 120.

Next, as illustrated in FIG. 5,

a groove 112 recessed by a predetermined depth from the upper portion of the coupling jig 110 to the lower portion of the coupling jig 110 may be defined in a central portion of a top surface of the coupling jig 110.

The groove 112 may horizontally extend by a predetermined length from one side of the coupling jig 110 to the other side of the coupling jig 110

while communicating the through hole 111.

An end of tools such as a flat-head screw driver may be inserted into the groove 112.

In this state, the tools such as the flat-head screw driver may be forwardly and reversely rotated to more easily screw-couple the lower portion of the coupling jig 110 to the accommodation groove 121 of the probe support 120.

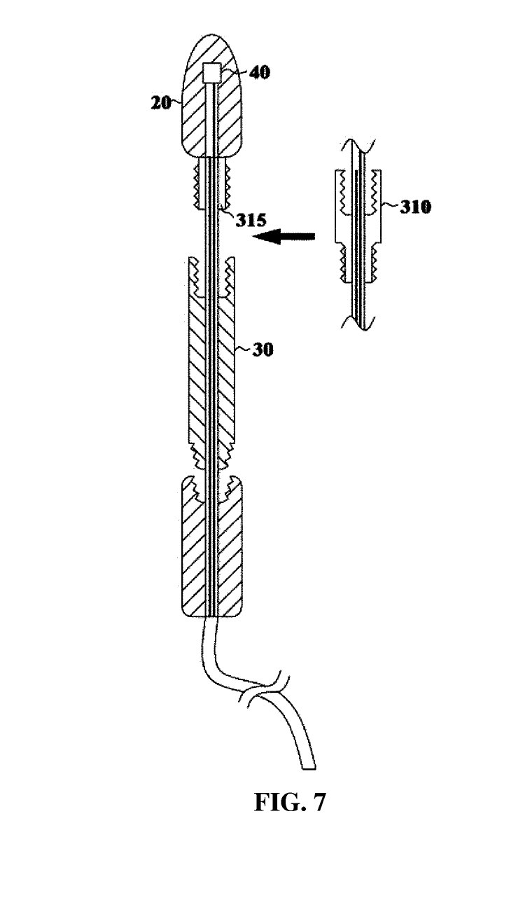

FIG. 6 is a schematic plan view of a cover 70.

Next, for sanitation of the insertion unit 10, the high-frequency output unit 20, and the electrode unit 30, which are inserted into the prostate tissue through an anus, and also to smoothly insert the insertion unit 10, the high-frequency output unit 20, and

the electrode unit 30 into the anus, a disposable cover 70, which is made of a synthetic resin such as polyisoprene and polyurethane, surrounding an outer circumferential surface of the upper portion of the probe support 120 of the insertion unit 10, an outer circumferential surface of the high-frequency output unit 20, and an outer circumferential surface of the electrode unit 30 may be provided.

FIG. 7 is a cross-sectional view of a high-frequency treatment device, which has a structure different from that of the high-frequency treatment device of FIG. 3 according to another embodiment, and FIG. 8 is a coupled cross-section view of FIG. 7.

FIG. 7 illustrates a high-frequency treatment device having an inner shape different from that of the high-frequency treatment device of FIG. 3. FIG. 7 is a view illustrating a state in which a high-frequency output unit is coupled to an electrode unit including a probe support by using a separate coupling jig 310.

Additionally explaining, the insertion unit 10 includes a coupling jig 310 coupled to a coupling member including a screw, which is disposed on a protrusion 315 disposed on a lower portion of the high-frequency output unit 20, and a coupling member disposed on an upper portion of the probe support or the electrode unit.

As described with reference to FIG. 7, even a case only the electrode unit is mentioned, the high-frequency treatment device may include the probe support 120 described in FIG. 3. Also, other components that are not separately described in FIG. 7 may be cited from those in FIG. 3.

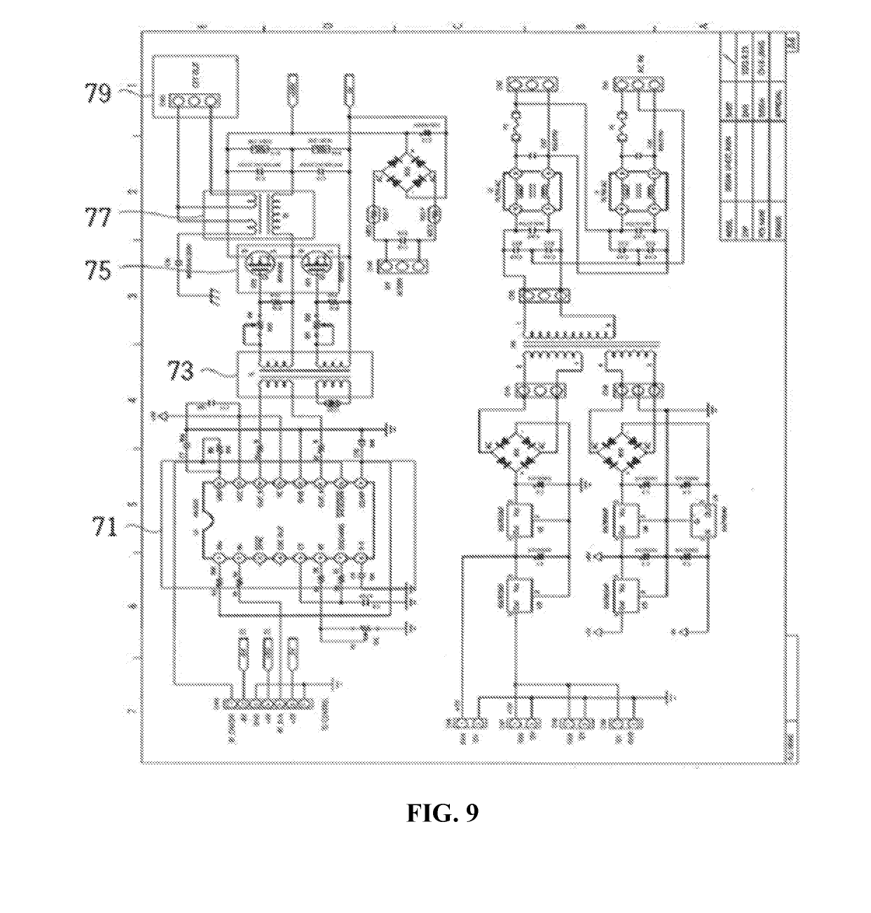

FIG. 9 is a view illustrating an example of a circuit generating the high-frequency current.

As illustrated in FIG. 9, a frequency generator 71, a first transformer 73 boosting up a voltage, an amplifier 75, a variable second transformer 77, and an output unit 79.

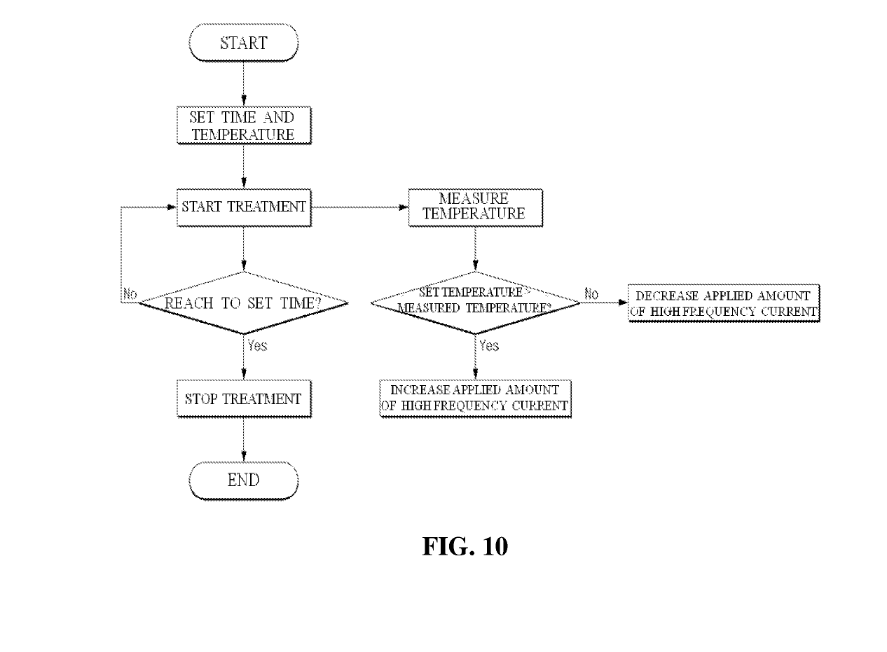

FIG. 10 is a schematic flowchart illustrating a process for treating prostate of the patient.

Next, as illustrated in FIG. 1, a temperature setting unit 50, a timer 60, and an operation switch 80 may be further provided on

a lower portion of a front surface of the main body 5 in various manners such as

a push button and a dial button so as to be exposed to the outside of the main body 5.

A reference temperature value of the high-frequency output unit 20 may be set through the temperature setting unit 50.

The high-frequency output unit 20 generates high-frequency deep heat

within a time range, which is set through the timer 60, for generating the high-frequency deep heat.

The reference temperature value of the high-frequency output unit 20 set through the temperature setting unit 50 and the time for generating the high-frequency deep heat set through the timer 60 may be displayed on a display unit 51 disposed on the upper portion of the front surface of the main body 5 by using a number, a character, or the like.

When the operation switch 80 is set to an ON state,

the main body 5 generates the high-frequency current. When the operation switch 80 is set to an OFF state, the main body 5 stops the generation of the high-frequency current.

When the time for generating high-frequency deep heat generated by the high-frequency output unit 20 exceeds

the time range, which is set by the timer 60, for generating high-frequency deep heat

in the state in which the operation switch 80 is set to the ON state, the main body 5 stops the generation of the high-frequency current

as illustrated in FIG. 7.

When the time for generating high-frequency deep heat generated by the high-frequency output unit 20 is within the time range, which is set by the timer 60, for generating high-frequency deep heat, the main body 5 continuously generates the high-frequency current.

When the temperature, which is measured by the temperature sensor 40, of the high-frequency deep heat of the high-frequency output unit 20 is below the reference temperature value that is set through the temperature setting unit 50, as illustrated in FIG. 7, the high-frequency current applied from the main body 5 to the high-frequency output unit 20 may increase in amount to raise the temperature of the high-frequency deep heat of the high-frequency output unit 20.

When the temperature, which is measured by the temperature sensor 40, of the high-frequency deep heat of the high-frequency output unit 20 is above the reference temperature value that is set through the temperature setting unit 50, the high-frequency current applied from the main body 5 to the high-frequency output unit 20 may decrease in amount to reduce the temperature of the high-frequency deep heat of the high-frequency output unit 20.

As described above, according to the present invention, the high-frequency output unit and the electrode unit may be provided to the insertion unit 10 to be inserted into the prostate tissue to allow the insertion unit 10, the high-frequency output unit 20, and the electrode unit 30 to be combined as one product, and also,

the patient's prostate tissue may be more efficiently treated by using the three components, i.e., the insertion unit 10, the high-frequency output unit 20, and the electrode unit 30.

### INDUSTRIAL APPLICABILITY

The present invention may be applied for the prostate treatment.

1. A high-frequency prostate treatment device comprising:
an insertion unit (10) inserted into a prostate tissue through an anus;
a high-frequency output unit (20) inserted into the insertion unit 10 to receive high-frequency current from a main body (5) that generates the high-frequency current, thereby generating high-frequency deep heat;
an electrode unit (30) disposed on the insertion unit (10) while being spaced a predetermined distance from the high-frequency output unit (20), the electrode unit (30) having a potential opposite to that of the high-frequency output unit (20).

2. The high-frequency prostate treatment device of claim 1, wherein the insertion unit (10) comprises:
a coupling jig (110) of which an upper portion is screw-coupled to the high-frequency output unit (20);
a probe support (120) in which an accommodation groove (121) accommodating a lower portion of the coupling jig (110) is defined in an upper portion thereof, and a through hole (122) communicating with the accommodation groove (121) vertically extends by a predetermined length so that the through hole (122) is defined below the accommodation groove (121), the probe support (120) being accommodated in the electrode unit (30) having a tubular shape; and
a fixing cap (130) screw-coupled to a lower portion of the probe support (120).

3. The high-frequency prostate treatment device of claim 2, wherein a temperature sensor (40) is disposed in the high-frequency output unit (20) so that the temperature sensor (40) is disposed above the coupling jig (110).

4. The high-frequency prostate treatment device of claim 2, wherein a through hole (111) is vertically and eccentrically defined in the coupling jig (110).

5. The high-frequency prostate treatment device of claim 4, wherein, since the through hole (111) of the coupling jig (110) is vertically and eccentrically defined in one side of the coupling jig (110), a lower portion of the other side of the coupling jig (110) has an area greater than that of a lower portion of one side of the coupling jig (110) to connect an upper portion of the other connecting line (9) except for one connecting line (7) for the temperature sensor to the lower portion of the other side of the coupling jig (110).

6. The high-frequency prostate treatment device of claim 2, wherein a groove (112) having a predetermined depth is defined in a top surface of the coupling jig (110).

7. The high-frequency prostate treatment device of claim 6, wherein the groove (112) horizontally extends by a predetermined length from one side of the coupling jig (110) to the other side of the coupling jig (110) while communicating with the through hole (111).

8. The high-frequency prostate treatment device of claim 2, wherein an accommodation groove (131) accommodating a lower portion of the probe support (120) is defined in an upper portion of the fixing cap (130), and a through hole (132) communicating with the accommodation groove (131) is defined in a lower portion of the fixing cap (130).

9. The high-frequency prostate treatment device of claim 1, further comprising:
a temperature setting part (50) setting a reference temperature value of the high-frequency output unit (20); and
a timer (60) setting a time range for generating high-frequency deep heat that is generated by the high-frequency output unit (20).

10. The high-frequency prostate treatment device of claim 1, further comprising a cover (70) surrounding the insertion unit (10), the high-frequency output unit (20), and the electrode unit (30).

11. The high-frequency prostate treatment device of claim 1, wherein, in a shape of the high-frequency output unit, the high-frequency output unit has a width that gradually decreases toward an upper end thereof, and the upper end of the high-frequency output unit has a width less than a transversal width of at least the electrode unit.

12. A high-frequency prostate treatment device comprising:
an insertion unit (10) inserted into a prostate tissue through an anus;
a high-frequency output unit (20) inserted into the insertion unit (10) to receive high-frequency current from a main body (5) that generates the high-frequency current, thereby generating high-frequency deep heat;
an electrode unit (30) disposed on the insertion unit (10) while being spaced a predetermined distance from the high-frequency output unit (20), the electrode unit (30) having a potential opposite to that of the high-frequency output unit (20),
wherein the insertion unit (10) comprises a coupling jig (310) coupled to a coupling member disposed on a protrusion disposed on a lower portion of the high-frequency output unit (20) and a coupling member disposed on an upper portion of the electrode unit or the probe support.
